(19) United States
(12) Patent Application Publication
(10) Pub. No.: US 2015/0306411 A1
(43) Pub. Date: Oct. 29, 2015

(54) APPARATUS AND METHOD FOR TREATMENT OF ORGANIC HUMAN TISSUE WITH A LOW PRESSURE PLASMA

(71) Applicant: NORTHCO VENTURES GMBH & CO. KG, Nürnberg (DE)

(72) Inventors: Josef SRB, Blatna (CZ); Josef KOROUS, Blatna (CZ); Jan HINTERKOPF, Aachen (DE)

(21) Appl. No.: 14/654,445

(22) PCT Filed: Oct. 21, 2013
(86) PCT No.: PCT/EP2013/003160

§ 371 Date: Jun. 19, 2015

(30) Foreign Application Priority Data
Dec. 21, 2012 (DE) 10 2012 025 079.7

(51) Int. Cl. A61N 1/44

(52) U.S. Cl. CPC A61N 1/44

(57) ABSTRACT

An apparatus for treating biological tissue (G) using a low-pressure plasma with
a) a transformer for generating a high-frequency electromagnetic field,
b) a probe, which can be electrically coupled to the transformer and
c) a control device for controlling the high-frequency electromagnetic field generated by the transformer,
   wherein a safety device, by which the power of the electromagnetic field generated by the transformer can be set automatically for the corresponding application, is associated with the control device.

The invention relates to an apparatus for treating biological tissue using a low-pressure plasma according to the preamble to claim 1. The invention further relates to a method for treating biological tissue using a low-pressure plasma.

It is known that plasmas have antimicrobial properties. The causes of the antibacterial effect of a plasma lie in heat, dehydration, shear stress, UV radiation, free radicals and charges. In the case of low-pressure plasmas, which are also called cold plasmas, heat plays a subordinate role, since these plasmas are operated at room temperature. In such low-pressure plasmas particularly reactive particles are produced, such as for example different oxygen or nitrogen species, which have a sufficiently long service life to damage organic compounds with indirect exposure. These particles include inter alia atomic oxygen, superoxide radicals, ozone, hydroxyl radicals, nitrogen monoxide and nitrogen dioxide. These particles exhibit a destructive effect on the most varied cell components.

If cell walls of bacteria, germs, viruses, fungi or other comparable microorganisms are directly exposed to the plasma, they become negatively charged by the bombardment with electrons present in the plasma. Due to the electrostatic repulsion this leads to mechanical stresses to the extent of exceeding the tensile strength and destruction of the cell wall. However, the cell walls can be destroyed not only by mechanical stresses due to the charge, but also by the disruption of the charge balance of the cell wall by different, further electrostatic interactions and by electrolysis, for example due to changing of the permeability of the cell walls. A mechanism for inactivation of microorganisms is also produced by the very high-energy ions, which may have more than 100 eV in capacitively coupled systems. Bombardment with such species can alter or destroy the structural integrity of the cells; however, a apparatus for generating such ion beams is complex and only suitable for treating living biological tissue, in particular human or animal tissues, with very high expenditure on apparatus.

Low-pressure plasmas are therefore particularly well suited for treatment of human or animal tissue, in particular skin surfaces, open wounds, the gums, the oral cavity or the like, in order to achieve disinfection of the tissue, in particular killing bacteria, germs, viruses, fungi or other comparable microorganisms which are located in or on the tissue.

An apparatus and a method for treating biological tissue with ozone is known from DE 10 2005 000 950 B3. This apparatus consists substantially of a transformer which can be adjusted in voltage and/or current intensity by means of a control device for generation of special directed voltage or current pulses having the most varied characteristic with or without a d.c. voltage component. In this case the d.c. voltage component is built up by additional electrodes on the biological tissue to be treated with the aid of an external voltage source or circuit. The primary coil of the transformer is the coil of a damped oscillating circuit through which high-frequency alternating current flows. Together with the capacitor to be charged, the secondary coil forms a resonant circuit of which the frequency corresponds to that of the transformer. A resonant transformer often serves as current source. Thus the treatment of different tissue types is possible, such as for example gums for treatments in the oral cavity or skin tissue for dermatological treatments with ozone. The setting of the power of the pulse generator takes place by means of operating elements which are disposed on the control device. By means of a rotary knob the power is set within a power range by the user or the attending doctor with reference to a numerical scale.

However, with this known apparatus an operating error is possible by which an incorrect power range is set. For example, for a dental treatment the power range for a dermatological application may be selected. This incorrect setting leads to a very painful treatment for the patient and possibly even to health problems.

Therefore the object of the invention is to modify an apparatus for treating biological tissue using a low-pressure plasma with the features of the preamble to claim 1 in such a way that such incorrect settings are ruled out. A further object of the invention is to provide a method for treating biological tissue using a low-pressure plasma by which a treatment is possible without flashover between the primary and secondary coil.

In terms of apparatus this object is achieved by an apparatus with all the features of claim 1. In terms of method this object is achieved by a method with all the features of claim 12. Advantageous embodiments of the invention are set out in the claims which are dependent upon the independent claims 1 and 11.

The apparatus according to the invention for treating biological tissue using a low-pressure plasma essentially comprises
a transformer for generating a high-frequency electromagnetic field,
a probe, which can be electrically coupled to the transformer and
a control device for controlling the high-frequency electromagnetic field generated by the transformer,
   wherein a safety device, by which the power of the electromagnetic field generated by the transformer can be set automatically for the corresponding application, is associated with the control device. By this special configuration of the apparatus according to the invention it is ensured that the safety device automatically sets the necessary and sensible treatment for the required treatment and thus operating errors which result in an increased power consumption of the transformer are avoided.

Advantageously the transformer comprises a transformer housing having a coupling which lies opposite a coupling for the probe for electrical/electronic connection of the control device, wherein the transformer housing is preferably constructed as a handle and is correspondingly ergonomically formed. This measure relates to a compact construction of the entire apparatus according to the invention, since both the transformer itself and also the control unit can be disposed inside the transformer housing. Only the probe for treatment of the biological tissue and, where appropriate, an external power source for supplying power to the apparatus according to the invention are not disposed inside the transformer housing. The ergonomic configuration of the transformer housing as a handle, which in its basic form is cylindrical, also enables pleasant and reliable handling of the apparatus according to the invention by the user.

Therefore for the reasons just given of compact construction and the simple, reliable and pleasant handling of the apparatus according to the invention, according to an advantageous idea of the invention the control device is disposed in the transformer housing.

However, for certain applications it may be sensible to dispose the control device outside the transformer housing. In particular when very delicate treatments have to be carried out, in which additional weight within the transformer housing designed as a handle is obstructive in the handling of the apparatus according to the invention.

The control device can be connected to an electrical power source so that the apparatus according to the invention can be supplied with the electrical power necessary for operation. In this case, in particular in the case of a control device disposed inside the transformer housing designed as a handle, a power source in the form of batteries or accumulators which is likewise accommodated in the transformer housing can, however, also be disposed outside the transformer housing. This is sensible in particular since the entire apparatus according to the invention can be operated independently of a stationary power source and in particular independently of a public or non-public electrical network. However, it is of course also conceivable to provide a stationary power source or a public or non-public electrical network as a power source to which the control unit can be connected.

According to a particularly advantageous concept of the invention, the safety device is designed as a jumper inside a coupling, the coupling being associated with the probe or the control device, or inside an electrical plug-type connection system between the probe and the transformer. In this case the jumper in the plug-type connection system acts as a type of switch, by which the power consumption of the control device or of the transformer can be fixed by the control device. Depending upon the position or location of the jumper, use is specified in the function of the tissue to be treated, for example gums or external treatment surface on the derma, for example an open wound. Advantageously there is no switch on the operating elements of the apparatus according to the invention so that an operating error can be avoided. Naturally such switches can also be provided, but these can only be actuated after a dialogue with the control device, wherein the user must confirm the change of power at least once. Furthermore it may be specified that such a change of power only takes place for an application over a specific time period, for example 30 seconds. After expiration of this time period a new confirmation of the change of power would have to take place. Advantageously such a function can be monitored by means of a processor which is integrated for example in the control device.

However, it is particularly advantageous if the safety device is designed as a processor and the safety function of the device according to the invention is controlled by means of software running in the processor.

According to a further concept of the invention the processor is designed to communicate with the probe. The probe then for example notifies the processor as to the applications for which it is designed, so that the processor prompts the control device to make the transformer generate only the power necessary for this probe. Thus in this respect the processor is also designed for determination of the power parameters for the control device and/or the transformer.

It has proved beneficial that the processor is designed for forwarding, in particular for encrypted forwarding of the power parameters for the control device and/or the transformer to the control device. Thus the processor can forward the power parameters which it has received through the communication with the probe directly and in encrypted form to the control device.

According to an independent concept of the invention the safety device is connected to a sensor for communication purposes, wherein the sensor determines the environmental parameters of a treatment surface of the probe, wherein the sensor in particular determines the composition of the atmosphere in the environment of the treatment surface of the probe. With reference to the composition of the atmosphere it is in fact possible to recognise whether a treatment in the oral cavity or on the skin of a patient is to take place.

Thus the invention enables an automatic adjustment of the necessary power range for an indication provided outside or inside the human body, without the need to carry out structural changes or uncontrolled adjustments on the device according to the invention. The individual converters or plasma generators according to the invention, which are used for different applications and power ranges, differ in the design of the control device. The control device regulates the necessary power automatically, without action by third parties and without harming the patient, but in accordance with the indication for successful treatment.

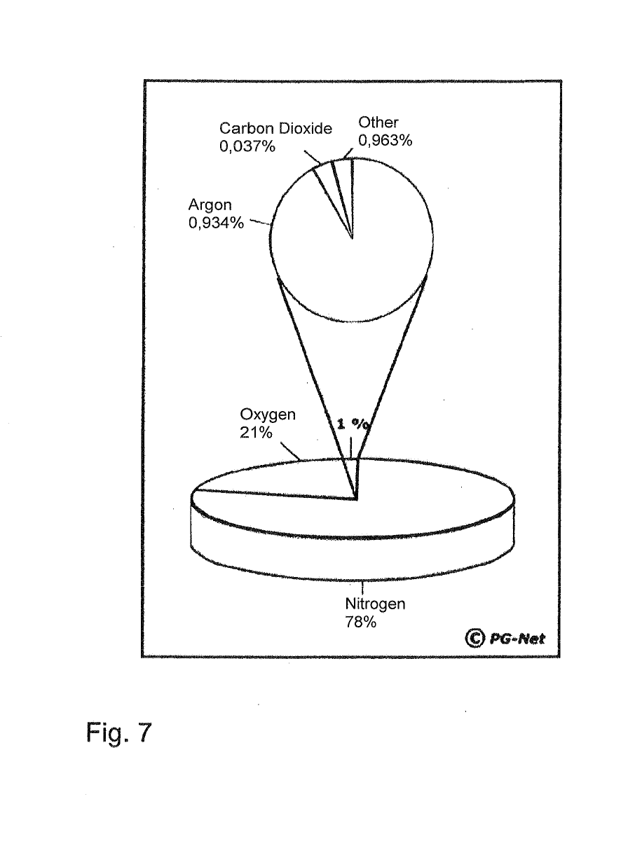

The great difference as to whether the use of the atmosphere outside or inside the human body can be used for a harmless and at the same time successful indication lies in the changed constituents of the atmosphere in the dry or moist state. This is illustrated in FIG. 7. This shows the composition of dry and pure atmospheric air at ground level.

The comparison in the table shows the changed constituents of the composition of the air in an oral cavity solely due to inhalation and exhalation.

| inspiratory fraction | gas | expiratory fraction^{[3]} |
|---|---|---|
| 78% | nitrogen | 79% |
| 21% | oxygen | 16% |
| 0.04% | carbon dioxide | 4% |
| 1% | noble gases | 1% |

In a further advantageous embodiment it would also be conceivable that the device has a measuring arrangement for analysing air, for instance a measuring arrangement which determines an oxygen content and/or a carbon dioxide content. This measurement may be indicative of the specific application and the pulses or the power can be set accordingly.

The probe by which the actual treatment is carried out is preferably constructed as a glass probe, since the necessary low-frequency plasma for application to the tissue to be treated is generated by the probe. Such glass probes are simple to handle and are physiologically harmless for application to or in biological tissue.

In this case it has proved worthwhile to fill the glass probe under underpressure, or partial vacuum, with respect to atmospheric pressure, preferably under underpressure from 500 Pa to a maximum of 3000 Pa, with a conductive gas, preferably with a noble gas or noble gas mixture. With such conductive gases, in particular noble gases and noble gas mixtures, preferably of argon and/or neon, the production of low-frequency plasmas and thus the entire apparatus according to the invention is particularly efficient. The glass probe is closed at one end by a metal contact, by which the high-frequency high voltage supplied by the transformer is conducted into the interior of the glass probe. Within the glass probe the gas is exposed to the high-frequency electromagnetic field and thus generates a glow discharge.

In this case the output of the transformer can be adjusted by the control device in such a way that voltages in the range between 1800 V and 35000 V can be set, which are transmitted to the treatment surface of the glass probe by means of the conductive gas inside the glass probe. If the treatment surface of the glass probe is located immediately above the biological tissue to be treated, this voltage is set between them, optionally as a function of the electrical resistance of the surface of the biological tissue to be treated and the resistance the gases, in particular the air, between the treatment surface of the glass probe and the surface of the biological tissue to be treated.

In order for the high-frequency high voltage provided by the transformer to be usable efficiently by the probe, a good and reliable electrical contact between the transformer and the probe is indispensable. According to an independent concept of the invention this is achieved in that the probe can be coupled electrically/electronically to the transformer by means of a contact spring. In this case it is conceivable that the contact spring is disposed on the transformer or the transformer housing. On the other hand the contact spring can also be disposed on the probe. In both cases the contact spring ensures the electrical contact between the probe and the transformer, even if an undesirable play occurs within the coupling between the probe and the transformer.

The method according to the invention for treating biological tissue using a low-pressure plasma with an apparatus as previously described essentially contains the following method steps:
a) providing electrical power in the form of electrical d.c. voltage or low-frequency a.c. voltage in the range from 12 V to 600 V with a current intensity on the side of the secondary coil from 0.1 μA to 300 μA,
b) converting the electrical d.c. voltage or the electrical low-frequency a.c. voltage into high-frequency a.c. voltage between 10 kHz and 50 kHz,
c) transforming the high-frequency a.c. voltage into a voltage range between 1800 V and 35000 V,
d) transmitting the high-frequency a.c. voltage in a voltage range between 1800 V and 35000 V to a probe (2), preferably a glass probe, which is positioned above the biological tissue to be treated with a spacing between 1 mm and 5 cm.

In this regard it is pointed out that in applications in the dental field, for example in the treatment of the gums in the oral cavity, the current intensity on the side of the secondary coil is chosen to be between 0.1 μA and 100 μA, whereas in applications to other tissue surfaces, in particular dermatological treatments of the rest of the skin or of the patient to be treated or gynaecological applications, the current intensity on the side of the secondary coil is chosen to be between 0.1 μA and 300 μA.

Further objects, advantages, features and possible applications of the present invention are apparent from the following description of embodiments with reference to the drawings. In this case all the features described and/or illustrated, considered alone or in any sensible combination, form the subject of the invention, also independently of their composition in the claims or their dependencies.

In the drawings:

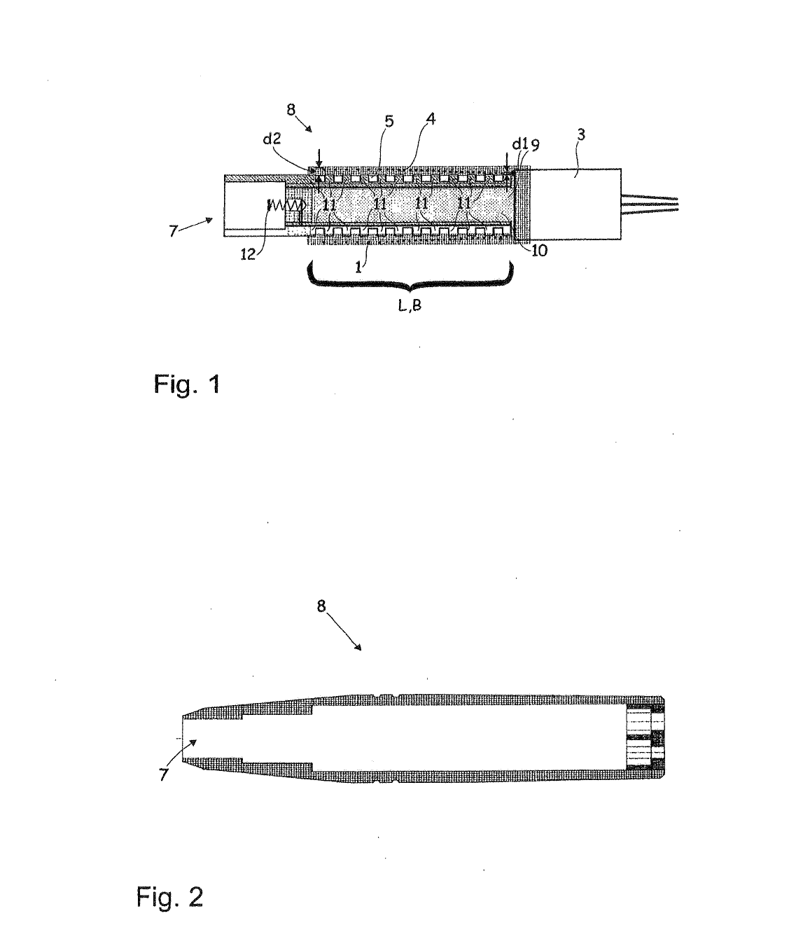

FIG. 1 shows a transformer of an embodiment of an apparatus according to the invention in a transformer housing,

FIG. 2 shows a transformer housing of an embodiment of an apparatus according to the invention,

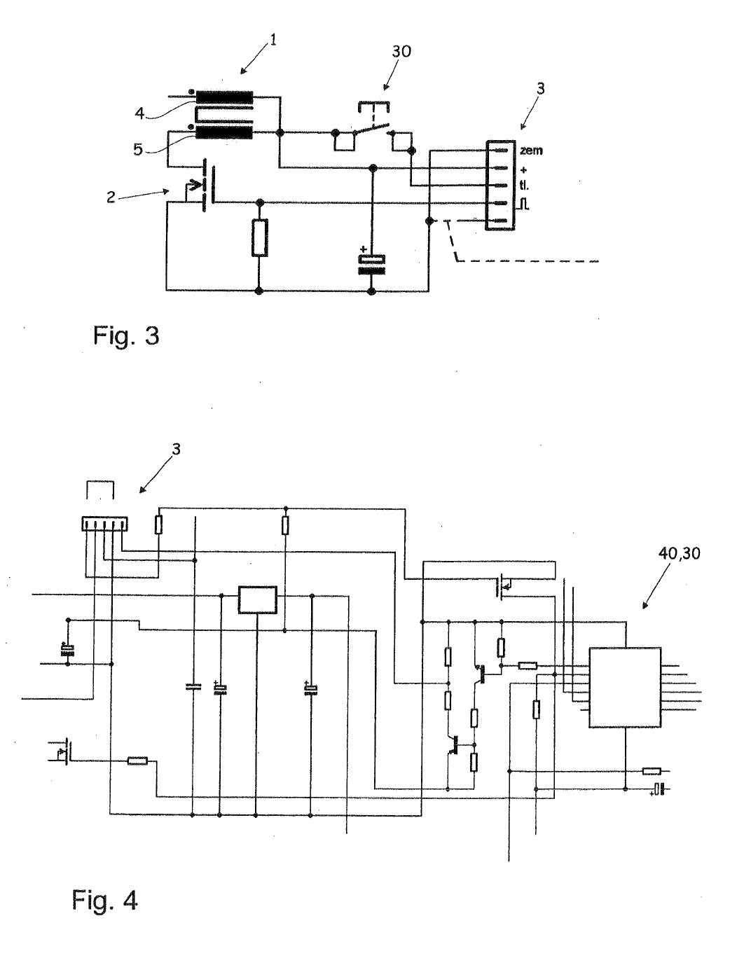

FIG. 3 shows a circuit diagram of an embodiment of an apparatus according to the invention,

FIG. 4 shows a further circuit diagram of an embodiment of an apparatus according to the invention,

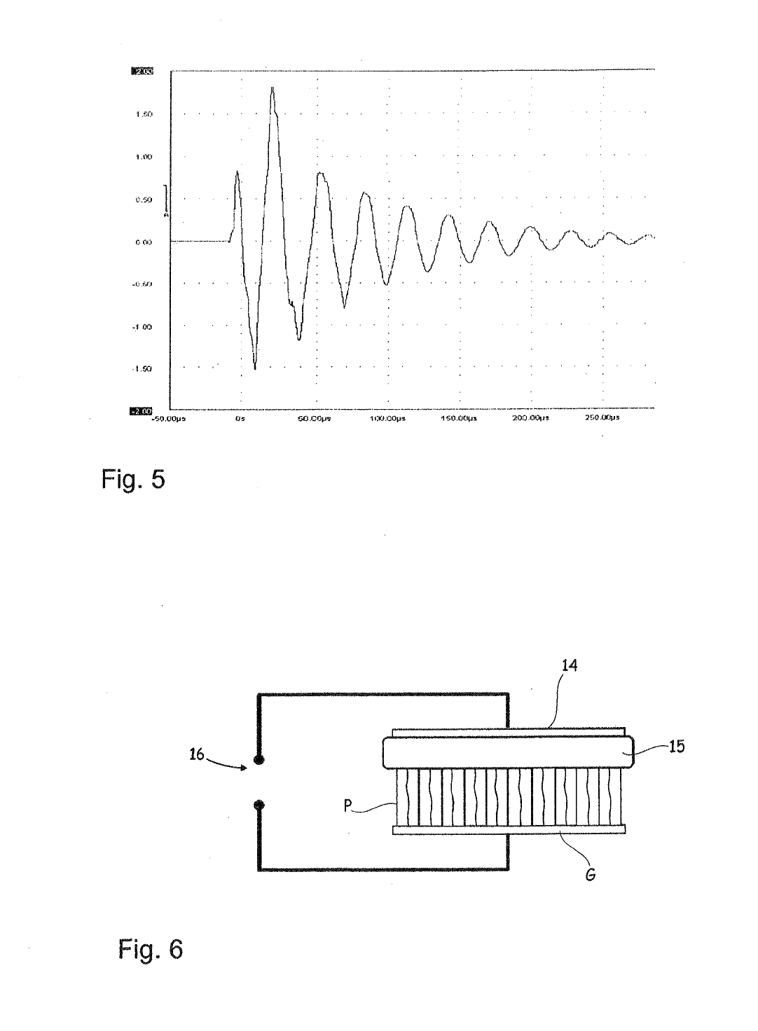

FIG. 5 shows a typical pulse pattern of a high-frequency voltage pulse, wherein the current intensity is shown in μA against the time,

FIG. 6 shows a schematic representation of a dielectric barrier discharge, and

FIG. 7 shows a representation of the composition of dry and pure atmospheric air at ground level.

In the drawings various elements of embodiments of apparatus according to the invention for treating biological tissue with a low-pressure plasma are shown which are explained in greater detail below.

FIG. 1 shows, for example, an embodiment of a transformer housing 8 of an apparatus according to the invention, in which a transformer formed from a primary coil 4 and a secondary coil 5 is disposed, a control device 3 being connected thereto via a coupling 9. The control device 3 in turn is connected to an electrical power source 13 (not shown here) for feeding electrical power into the transformer 1. A coupling 7 on which a probe 2, preferably a glass probe, can be disposed is in turn disposed on the end of the transformer housing 8 opposite the coupling 9. In this case a contact spring 12 ensures that an electrical contact always exists between the transformer 1 and the probe 2. In the present case the transformer housing 8 is constructed as a handle and extends in its longitudinal extent in the same direction as the primary coil 4 and the secondary coil 5.

In this embodiment the secondary coil 5 is wound around a rod core 10 which is preferably made of a ferrite, whereas the primary coil 4 is wound with a spacing around the secondary coil 5. This spacing increases continuously from the end of the coils 4 and 5 facing the coupling 9 with a spacing d1 to the end of the coils 4 and 5 facing the coupling 7 up to a spacing d2, so that the primary coil 4 is disposed conically coaxially over the secondary coil. In the present embodiment both coils 4 and 5 have the same length L, so that they form an overlap region B over their entire length. In this case the primary coil 4 also takes on the function of an electromagnetic shield, or ensures a shielding effect, by which electromagnetic interference fields cannot critically disrupt the high-frequency electromagnetic field generated by the transformer 1, so that satisfactory functioning of the apparatus according to the invention is provided. In addition, sealing means can also be provided in an end section of the converter.

In this embodiment the transformer 1 constructed as a high-voltage transformer is designed in such a way that the inner secondary coil 5 is wound around a rod core 10 made of ferrite in chambers 11. In the embodiment shown here the secondary coil 5 has 500 turns per chamber 11; however, other numbers of turns are also conceivable.

On the one hand the transformer 1 takes on the task of converting the high-frequency low voltage supplied by the power source 13 and the control unit 3 into a high-frequency high voltage. On the other hand, however, it also takes on the task of conducting the generated high voltage in particular via a glass tube (not shown here) of the probe 2 constructed as a glass probe to the treatment surface thereof which is disposed on the end of the probe opposite the coupling 7.

The arrangement of the coils 4 and 5 inside the transformer 1 leads to the provision of pulses with a predetermined signal form, preferably of sinusoidal pulses and particularly preferably of exponentially damped sinusoidal pulses, such as are illustrated for example in FIG. 5 and with which a cold plasma or a low-pressure plasma can be generated between the treatment surface of the probe 2 and the tissue to be treated.

FIG. 2 shows the structure of a transformer housing 8 of FIG. 1, which is produced from an electrically insulating material, preferably a plastic.

On the end of the transformer housing 8 having the coupling 7 for the probe 2, said housing is equipped with a contact spring 12 which is electronically connected to the transformer 1. As already mentioned briefly, the contact spring 12 produces the contact with the probe 2. The voltage pulses are transmitted to the probe 2 by the contact. The probe 2 constructed as a glass probe is equipped in the usual way with two chambers. The inner chamber is preferably gas-filled with 100% neon at a negative pressure of 500 Pa to a maximum of 3000 Pa and conducts the high voltage to the tip of the instrument probe. The outer chamber serves for insulation and protection of the inner chamber. The inner chamber is advantageously made of glass and the outer chamber can be made of the materials glass or precious metal.

At the end opposite the treatment surface the probe 2 is closed by a metal flap which together with the contact spring 12 and the coupling 7 produces the electrical plug-type connection system with the transformer 1 disposed in the transformer housing 8.

Between the treatment surface of the probe 2 and the biological tissue G to be treated, with a spacing between 1 mm and 5 mm, the supplied high-frequency a.c. voltage and the typical pulse pattern produce the formation of the cold plasma or of the low-pressure plasma by which bacteria, germs, viruses, fungi or other comparable microorganisms adhering to the tissue G can be killed.

The gas in the probe 2 constructed as a glass probe is exposed to the generated high-frequency, electromagnetic alternating field in order to generate a glow discharge (microdischarge). In this case the output of the transformer can be adjusted via the control device 3 in such a way that voltages in the range between 1.8 V and 35 V can be set, which are transmitted to the treatment surface of the probe 2 by means of the conductive gas. If the treatment surface of the probe 2 is located immediately above the tissue G to be treated, the voltage thereof is set as a function of the skin resistance of the air between the instrument probe tip and the skin surface.

The method for direct generation of a low-pressure plasma or cold plasma corresponds to the structure of the dielectric barrier discharge illustrated in FIG. 6. The excitation voltage is generated in the transformer 1. In this case the probe 2 forms a metal electrode 14 and a dielectric 15. The earth electrode is formed by the tissue G to be treated, so that between the tissue G and the metal electrode 14 of the probe 2 substantially the high-frequency excitation voltage 16 supplied by the transformer 1 is applied. The illustrated diagram serves as a model for other assessments.

Physical assessment of the plasma formation by dielectric barrier discharge. The dielectric barrier discharge, also called dielectrically hindered discharge or silent discharge, causes non-thermal plasma filaments P at atmospheric pressure during the ignition phase. In this assessment the dielectrically hindered discharge or silent discharge is, alongside corona discharge, a variant of the gas discharges which cause non-thermal plasma filaments P at atmospheric pressure during the ignition phase. The difference between the two forms of gas discharge lies in the extinguishing mechanism of the discharge filaments. In the case of the corona discharge it is space charge-oriented and in the case of the barrier discharge it is surface charge-oriented.

The principal structure illustrated in FIG. 6 consists of two electrodes, a high-voltage electrode 14 and an earth electrode G, with one or more dielectric barriers 15 (isolators) between them. A gap which is variable in width, of the order of magnitude of several mm to within the cm range, is located between the dielectric 15 and the earth electrode G. The sample to be treated is located on or forms the earth electrode G. In order to produce the discharge, an a.c. voltage of 1-100 kV and frequencies of 10-50 kHz are required. This discharge is characterised by the formation of microdischarges or plasma filaments P. In this reaction charge carriers accumulate on the surface of the dielectric 15 and weaken the external electrical field, which leads to extinguishing of the plasma filaments P. The dielectric 15 serves for current limitation and makes it possible for the discharges to take place at a plurality of statistically uniformly distributed points, thus enabling an areal plasma treatment of the entire surface of the tissue G to be treated.

FIGS. 3 and 4 show two circuit diagrams by way of example in which a safety device 30 is illustrated. Whilst the safety device according to FIG. 3 is designed as a contact bridge which carries out the setting of the power of the control device 3 or of the transformer 1 merely with reference to the position of the switch of the contact bridge, the configuration according to FIG. 4 is more complex.

In FIG. 4 the safety device 30 is implemented by a processor 40 which performs the setting of the power for the control device 3 or the transformer 1 under software control with the aid of the probe 2 installed in the apparatus according to the invention. In this case the probe 2 communicates with the processor 40 and gives it its application or operating parameters. With reference to this information the processor 40 now ascertains the appropriate power parameters for the control device 3 or the transformer 1 and communicates these to the control device 3. This, in turn, then makes the corresponding setting, so that the treatment can be carried out accordingly.

The physical assessment of the plasma formation takes place according to the Paschen and Townsend method. The analysis relates to the model for the dielectric barrier discharge illustrated in FIG. 6. The assessment makes it possible to determine the breakdown voltage (=ignition voltage) which leads to the formation of a plasma. Below the breakdown voltage plasma filaments P are present which are characteristic for a cold plasma or low-pressure plasma.

The starting point is a capacitor with a plate spacing of d=1 mm. Air is situated between the plates thereof. Let a be the probability per unit of length that an electron ionises a neutral atom or molecule, wherein impacts of ions with neutral atoms can be disregarded because of the rapidly changing field and the large mass of the ions.

If N is the number of electrons produced, then the following applies:

dN/dx=aN (1.1)

=>N(d)=Nₒe^{αd} (1.2)

Where N₀ is the number of externally generated electrons, for example by cosmic radiation. The number of ionising impacts is proportional to the pressure p and to the probability for an ionisation impact.

Moreover for the kinetic energy of the electrons the following applies:

Eᵢₒₙ=eEλᵢₒₙ (1.3)

Where λᵢₒₙ is the acceleration path and E is the applied electrical field strength. Because of inelastic impacts only a fraction

$$exp ( \frac{{λ}_{ion}}{{λ}_{inel}} )$$

runs through the path λᵢₒₙ without energy loss.

It follows for the constant α

$$α = {Ape}^{xp - ( \frac{{λ}_{ion}}{{λ}_{inel}} )} = Apexp - ( B \frac{p}{E} ) \qquad ( 1.4 )$$

With the breakdown voltage U_{zünd}=Ed the following is obtained:

$${U}_{zuend} = \frac{Bpd}{In ( Apd ) - In ( In ( 1 + {γ}^{- 1} ) )} \approx 3 kV \qquad ( 1.5 )$$

Where γ is the number of generated electrons per ion (third Townsend coefficient), with which the ignition condition

γ(e^{αd}−1)≧1 (1.6)

is produced. In this case generally y<<1 applies

The Paschen curve describes the dependence of the breakdown voltage for the generation of a gas discharge upon the product of gap size and pressure.

For the present case the dependence of the breakdown voltage upon the gap widths can be estimated

| gap width | U_{zünd} |
|---|---|
| 1 mm | 3 kV |
| 2 mm | 6 kV |
| 3 mm | 9 kV |
| 4 mm | 12 kV |
| 5 mm | 15 kV |
| 6 mm | 18 kV |

Thus the electrical breakdown occurs at a voltage of 3 kV for air at 1 bar. Since all atoms or molecules are ionised here on the entire path d, this is the upper limit for the voltage which is necessary for a stable plasma. Below this voltage, in a barrier discharge thin discharge channels (plasma filaments P) which are characteristic for a cold plasma form between the electrodes (spacing in the region of 1 mm). At atmospheric pressure, statistically distributed, a large number of transient discharge channels (microdischarges) are observed.

A necessary criterion for the existence of a plasma is that the Debye length is small by comparison with the measurements of the system. This shielding length is characterised in that on this length the potential of a local ion or electron discharge has fallen sufficiently dramatically (generally to 1/e times). This is therefore because in a plasma a positive ion is surrounded by a spherical cloud of electrons, so that the charges compensate each other to some extent, wherein the radius of these spheres is the Debye length. In the present case the movement of the ions in the alternating field relative to that of the electrons may be disregarded because of the much greater mass of the ions. The same applies to the Debye length.

$${λ}_{d} = \sqrt{\frac{{∈}_{o} {k}_{b} {T}_{e}}{{n}_{e} {e}^{2}}} \qquad ( 2.1 )$$

For a non-isothermal plasma, in which because of their smaller mass the electrons have a higher temperature than the ions, in the case of a barrier discharge

Tₑ˜−10 eV (2.2)

(electron temperature) and

nₑ˜10²⁰−10²¹m⁻³ (2.3)

(volume number density of the electrons).
If these values are inserted into the equation (2.1), then for the Debye length of a non-isothermal plasma of a barrier discharge

λ_{d}=2.35·10⁻⁶m (2.4),

wherein this Debye length was calculated for the most unfavourable case of a number density of nₑ=1020 M⁻³ and an electron temperature of Te=10 eV=1, 16·105 K.

If it is assumed for the present case that the system is of an order of magnitude in the mm range, then the Debye length is smaller by a factor of 1000, whereby the necessary criterion for the existence of a plasma is met.

A further criterion is that the average number of charged particles in the Debye sphere is greater than one. In the unfavourable situation nₑ=1020 m⁻³ approximately 5000 charged particles are situated in the Debye sphere, whereby this criterion is also met.

The parameters of the apparatus according to the invention meet the physical prerequisites for generating a cold plasma.

| | | | necessary |
|---|---|---|---|
| | | | condition |
| physical parameter | necessary condition | plasmaOne | met? |
| breakdown voltage | 3 kV with 1 mm gap | 3 to 18 kV | yes |
| Debye length | gap size >> | gap size >=1 mm | yes |
| | λ_{d} = 2.35 · 10⁻⁶ m | | |
| average number | number >1 | number: approx. | yes |
| of charged | | 5000 | |
| particles in | | | |
| Debye sphere | | | |

### List of Reference Signs

1 transformer
2 probe
3 control device
4 primary coil
5 secondary coil
7 coupling
8 transformer housing
9 coupling
10 rod core
11 chamber
12 contact spring
13 power source
14 metal electrode
15 dielectric
16 excitation voltage
30 safety device
40 processor
P plasma filaments
B overlap region
d1 spacing
d2 spacing
F finger
K total capacitance
CF capacitance finger
L length
SK resonant circuit
G tissue

1-12. (canceled)

13. An apparatus for treating biological tissue (G) using a low-pressure plasma with
a) a transformer for generating a high-frequency electromagnetic field,
b) a probe, which can be electrically coupled to the transformer and
c) a control device for controlling the high-frequency electromagnetic field generated by the transformer,
wherein a safety device, by which the power of the electromagnetic field generated by the transformer can be set automatically for the corresponding application, is associated with the control device.

14. The apparatus according to claim 13, wherein the transformer comprises a transformer housing having a coupling which lies opposite a coupling of the probe for electrical/electronic connection of the control device, wherein the transformer housing is preferably constructed as a handle and is correspondingly ergonomically formed.

15. The apparatus according to claim 13, wherein the control device is disposed inside or outside the transformer housing and can be connected to an electrical power source.

16. The apparatus according to claim 14, wherein the safety device is designed as a jumper inside an electrical plug-type connection system associated with the couplings between the probe or control device and the transformer.

17. The apparatus according to claim 13, wherein the safety device is constructed as a resistor, memory chip or processor.

18. The apparatus according to claim 13, wherein the memory chip or the processor is constructed to communicate with the probe.

19. The apparatus according to claim 13, wherein the memory chip or the processor is constructed to determine the power parameters for the control device and/or the transformer.

20. The apparatus according to claim 13, wherein the memory chip or the processor is designed for forwarding, in particular for encrypted forwarding of the power parameters for the control device and/or the transformer to the control device.

21. The apparatus according to claim 13, wherein the safety device is connected to a sensor for communication purposes, wherein the sensor determines the environmental parameters of a treatment surface of the probe, wherein the sensor in particular determines the composition of the atmosphere in the environment of the treatment surface of the probe.

22. The apparatus according to claim 13, wherein the probe is constructed as a glass probe.

23. The apparatus according to claim 22, wherein the glass probe is filled under negative pressure, preferably under negative pressure of 500 Pa to 3000 Pa, particularly preferably of 2000 Pa, with a conductive gas, preferably with a noble gas or noble gas mixture.

24. A method for treating biological tissue (G) with a low-pressure plasma with an apparatus according to claim 13, with the following method steps:
a) providing electrical power in the form of electrical d.c. voltage or low-frequency a.c. voltage in the range from 12 V to 600 V with a current intensity on the side of the secondary coil (5) from 0.1 μA to 300 μA,
b) converting the electrical d.c. voltage or the electrical low-frequency a.c. voltage into high-frequency a.c. voltage between 10 kHz and 50 kHz,
c) transforming the high-frequency a.c. voltage into a voltage range between 1800 V and 35000 V,
d) transmitting the high-frequency a.c. voltage in a voltage range between 1800 V and 35000 V to a probe, preferably a glass probe, which is positioned above the biological tissue to be treated with a spacing between 1 mm and 5 cm.
